(19) United States
(12) Patent Application Publication
(10) Pub. No.: US 2026/0263287 A1
(43) Pub. Date: Sep. 10, 2026

(54) METHOD AND APPARATUS FOR FORMING ABSORBENT SANITARY ARTICLES

(71) Applicant: GDM S.P.A., Bologna (IT)

(72) Inventors: Alessandro ZAVALLONI, Bologna (IT); Matteo PIANTONI, Bologna (IT)

(21) Appl. No.: 19/133,015

(22) PCT Filed: Nov. 29, 2023
(86) PCT No.: PCT/IB2023/062007

§ 371 Date: May 27, 2025

(30) Foreign Application Priority Data
Nov. 30, 2022 (IT) 102022000024672

(51) Int. Cl. A61F 13/15

(52) U.S. Cl. CPC A61F 13/15764; A61F 13/15723; A61F 13/15747; A61F 2013/15861

(57) ABSTRACT

Described is a method for forming an absorbent sanitary article made from a composite web elasticized at least along a machine direction of the composite web and including a succession of absorbent pads of corresponding absorbent sanitary articles; the forming method includes performing a process on the composite web at least partly wrapped round a rotary device; relaxing the composite web before performing a process on the composite web (W) so that the composite web is shortened until the absorbent pads of the absorbent sanitary articles and the corresponding absorbent sanitary articles are spaced at a working spacing of the rotary device.

### TECHNICAL FIELD

This invention relates to a method and an apparatus for forming absorbent sanitary articles from a continuous web.

### BACKGROUND ART

Commonly used in processes for forming absorbent sanitary articles are rotary devices on which the continuous web is wrapped; the continuous web is generally also called “composite web” in that it comprises a plurality of components which are joined to each other so that it can be subjected to different operations, such as, for example, welding. perforation, division into segments, folding, whereby the end product is formed while the rotary device rotates.

Generally speaking, the reference rotary devices involve using a rotary means supporting a plurality of working units or tools configured to perform the corresponding process, such as a welding, a folding or other process while the rotary means rotates about its axis of rotation.

The working units are angularly spaced from each other around the axis of rotation and the angular distance between one working unit and a working unit adjacent to it determines the working spacing on the continuous web.

The radial positioning of the working units relative to the axis of rotation of the rotary means determines the angular distance and the working spacing of the working units.

The working spacing is set by geometrical and/or constructional parameters of the end product to be made, and to form products with different characteristics, in particular different sizes, it is necessary to modify the working spacing.

Varying the working spacing of the rotary device can be done, for example, by varying the radial positioning of the working units relative to the axis of rotation of the rotary means so as to modify the angular distance between the working units.

Document EP3078479A1 describes a welding wheel provided with systems for attaching the welders to the rotary means and which allow the radial positioning of the welders to be modified.

In other solutions, the working spacing of the working units can be varied to form products of different sizes by extremely complex rotary means, such as, for example, the one described in document EP3936312A1, where the radial position of the working units can be modified continuously.

Alternatively, the rotary devices can be replaced with others where the angular distance between the working units is different, or it is possible to replace some of the components of the rotary devices such as, for example, in the solution described in EP2135591A1, which requires a great deal of time and resources.

In this context, there is a need for a method and an apparatus for making absorbent sanitary article to be versatile and to allow producing and forming products of different sizes in a manner that is simple and inexpensive compared to the prior art solutions.

### AIM OF THE INVENTION

This invention has for an aim to provide a method and an apparatus for forming nappies and capable of meeting the aforesaid need.

This aim is achieved by a method for forming and folding absorbent sanitary articles comprising the technical features set out in claim 1, and by an apparatus for forming and folding absorbent sanitary articles comprising the technical features set out in claim 11. The dependent claims correspond to possible different embodiments of the invention.

According to an aspect, this invention relates to a method for forming an absorbent sanitary article.

The absorbent sanitary article is made from a composite web which is elasticized at least in a feed direction of the composite web itself.

The composite web comprises a succession of absorbent pads of corresponding absorbent sanitary articles. The pads are disposed along a direction substantially orthogonal to the feed direction of the composite web.

The forming method comprises wrapping the composite web at least partly round a rotary device.

The forming method comprises performing a process or operation on the composite web which is at least partly wrapped round the rotary device. Preferably, the rotary device comprises a rotary means rotating in a feed direction of the web and a plurality of working units configured to perform the process while the rotary means rotates about an axis of rotation.

The working units are supported by the rotary means and are angularly spaced from each other about the axis of rotation of the rotary means. An angular distance between a first working unit and a second working unit adjacent to it determines a working spacing of the rotary device on the composite web. The forming method comprises relaxing the composite web before performing a process on the composite web so that the composite web is shortened until the absorbent pads of the absorbent sanitary articles and the corresponding absorbent articles are spaced at the working spacing of the rotary device.

The composite web is elasticized in the feed direction. The tension of the elastics causes the shortening, or relaxation, of the composite web.

For example, the rotary device may have the working units at the working spacing corresponding to the smaller size of the absorbent articles which can be formed and the elasticized webs of the larger sizes can be relaxed so that the absorbent articles move closer together until having the common spacing corresponding to the smaller spacing.

In practice, the working units are placed on the rotary device at a preset spacing and the step of relaxing is used to adjust the tension of the composite web so that the absorbent articles are brought to the preset spacing, which becomes the common spacing for all the sizes.

Preferably, relaxing the composite web comprises slowing down the composite web before performing a process on the composite web.

When the web slows down, the elastics in it tend to become shorter, so that, for example, the pads move closer together and tend toward the preset spacing.

Preferably, relaxing the composite web comprises a first relaxing of the composite web and a second relaxing of the composite web after the first relaxing of the composite web.

In practice, relaxing the composite web is preferably carried out in at least two distinct steps.

Relaxing the web in successive steps, for example in the case where the web needs to be significantly shortened, may allow shortening to be more controlled so that the preset spacing is reached more precisely.

For example, relaxing the web may occur on one or more relaxing suction wheels which slow down the web.

Performing a process may comprise separating the individual absorbent sanitary articles from the composite web.

Performing a process may comprise folding the individual absorbent sanitary articles.

The method may be adopted, for example, to form nappies of the type also known as “refastenable” nappies which, when open, are basically in the shape of a T. In this type of nappy, the elastic waistband or belt is the horizontal portion of the T and the crotch, comprising the absorbent pad, corresponds to the stem portion of the T. The crotch portion is folded towards the waistband, both in use and during manufacturing, while the end portions of the waist are in turn folded towards the centre line and fixed to the folded crotch portion.

A special feature of this type of nappy is that it can be worn like pants, by inserting the legs into the corresponding openings but, when necessary, it can be adjusted at the waist like a traditional nappy.

In effect, the end portions of the waist are fixed to the outside of the crotch portion by releasable fastening means.

Considering an absorbent article comprising a waistband having an intermediate portion from which the absorbent pad extends to separate a first and a second flap of the waistband, and comprising a first closing element at one end of the first flap and a second closing element at one end of the second flap, the forming method may comprise applying the succession of absorbent pads on the composite web so they are spaced from each other and project outwards from the web, folding the absorbent pads onto the composite web, applying a succession of the first closing elements on the composite web, applying a succession of the second closing elements on the composite web so that each absorbent pad is interposed between one of the first closing elements and one of the second closing elements.

The method may comprise temporarily fastening the folded absorbent pads to the composite web.

The step of temporarily fastening the folded absorbent pads to the composite web may comprises a step of plastically deforming at least one portion of each folded absorbent pad and one portion of the composite web at the portion of each folded absorbent pad to create mechanical interference between the absorbent pads and the composite web.

Preferably, the forming method comprises making a succession of lines of weakness in the composite web between each second closing element and the first closing element following it in the feed direction of the composite web.

The lines of weakness may be, for example, discrete cuts into the width of the composite web.

The step of making a succession of lines of weakness in the composite web between each second closing element and the first closing element following it in the feed direction of the composite web may be carried out at the same time as the composite web is relaxed during the step of relaxing the composite web.

Each line of weakness is made and disposed between consecutive absorbent articles in the feed direction of the composite web.

Performing a process on the composite web may comprise folding the first flap of each absorbent article towards the corresponding absorbent pad and folding the second flap of each absorbent article towards the corresponding absorbent pad.

Folding the first flap of each absorbent article towards the corresponding absorbent pad or folding the second flap of each absorbent article towards the corresponding absorbent pad may tear the composite web at the lines of weakness, thereby separating the absorbent articles from each other and from the composite web.

In the absorbent articles, the absorbent pad is, at least in use, folded towards the waistband to define a crotch portion of the absorbent article itself and the first and second flaps are folded onto the folded absorbent pad and fixed thereto by the first closing element and the second closing element, respectively, to define a pant-like article.

Advantageously, relaxing the composite web, for example in the case of forming absorbent articles of the larger size, hence in the case of a long composite web, allows bringing the absorbent pads and the lines of weakness to the preset working spacing of the rotary device so that separating and folding the individual absorbent articles are carried out precisely.

Folding the first flap of each absorbent article towards the corresponding absorbent pad may be carried out before folding the second flap of each absorbent article towards the corresponding absorbent pad.

Folding the first flap of each absorbent article towards the corresponding absorbent pad may be carried out after folding the second flap of each absorbent article towards the corresponding absorbent pad.

Folding the first flap of each absorbent article towards the corresponding absorbent pad may be carried out simultaneously with folding the second flap of each absorbent article towards the corresponding absorbent pad.

Folding the first flap of each absorbent article towards the corresponding absorbent pad comprises attaching the first flap to the corresponding absorbent pad with the first closing element.

Folding the second flap of each absorbent article towards the corresponding absorbent pad comprises attaching the second flap to the corresponding absorbent pad with the second closing element.

Performing a process may comprise welding the composite web. In such a case, the working units are provided with welders.

The absorbent articles which can be made are those known as “pant style nappies” which look and are worn like pants, with elastic waistbands which are welded or joined by a permanent connection.

Performing a process comprises welding the elastic gathers which encircle the waist of the wearer of the pant style nappy.

Advantageously, relaxing the composite web, for example in the case of forming pant style absorbent articles of the larger size, hence in the case of a long composite web, allows bringing the absorbent pads and the side gathers to the preset working spacing of the rotary device so that welding the individual absorbent articles is carried out precisely to close the waist of the nappies.

An absorbent article forming plant can also work at high speeds with all sizes which are, instead, slowed down when nearing the rotary device to bring them to its working spacing. That way, it is no longer necessary to adapt the rotary device to the spacing of the absorbent pads, with evident saving of time and resources.

The forming method may comprise preparing the continuous web and feeding it in the feed direction.

According to an aspect, this invention relates to an apparatus for forming an absorbent sanitary article made from a composite web which is elasticized at least in a feed direction of the composite web itself.

The composite web comprises a succession of absorbent pads of corresponding absorbent sanitary articles. The pads are disposed along a direction substantially orthogonal to the feed direction of the composite web.

The forming apparatus comprises a rotary device comprising a rotary means rotating in a feed direction of the composite web and a plurality of working units configured to perform a process on the composite web.

The working units perform the process on the composite web at least partly wrapped round the rotary device while the rotary device rotates about an axis of rotation.

The working units are supported by the rotary means and are angularly spaced from each other about the axis of rotation of the rotary means.

The angular distance between a first working unit and a second working unit adjacent to it determines the working spacing of the rotary device on the composite web. The working spacing depends on the size of the absorbent article to be made, to which the length of the composite web is also linked.

The forming apparatus comprises at least one first relaxing wheel for relaxing the composite web and disposed upstream of the rotary device.

The forming apparatus comprises a computerized command and control unit in communication with the first relaxing wheel.

The computerized command and control unit is configured to adjust a rotation speed of the first relaxing wheel as a function of the working spacing of the rotary device so that the composite web is shortened on the first relaxing wheel until the absorbent pads of the absorbent articles and the corresponding sanitary articles are spaced at the working spacing of the rotary device.

The relaxing wheel decreases the speed of the web relative to the speed of the web itself upstream of the relaxing wheel.

Using a slower wheel, the elastics of the composite web relax, hence pulling less, so that the products withdraw naturally, thus modifying the spacing of the products being processed.

The forming apparatus may comprise a second relaxing wheel between the first relaxing wheel and the rotary device. The computerized command and control unit is in communication with the second relaxing wheel and may be configured to adjust the rotation speed of the second relaxing wheel as a function of the working spacing of the rotary device.

The computerized unit is configured to adjust the rotation speed of the first and second relaxing wheels so that the composite web on the first and second relaxing wheels is shortened until the absorbent pads of the absorbent articles and the corresponding absorbent articles have the same working spacing as the rotary device.

The presence of more than one relaxing wheel allows reducing the speed of the web relative to the speed of the web upstream of the relaxing wheels in successive steps upstream of the rotary device.

Slowing and relaxing the web first on one wheel and then further on the next wheel allows greater control over the web and over product spacing.

The rotary device may be a folding wheel for folding the absorbent sanitary articles and the working units may each comprise at least one folder.

Each working unit may comprise a main support for receiving a respective absorbent pad of the succession of absorbent pads.

Each working unit may comprise a first and a second side support disposed on opposite sides of the main support to receive, respectively, a first and a second flap of a waistband of the absorbent sanitary article corresponding to the absorbent pad received by the main support.

The first and the second flap extend on opposite sides of the respective absorbent pad. Each working unit comprises a first and a second folder for folding the first side support and the second side support on the main support respectively.

The forming apparatus may comprise a cutting station for cutting the composite web, disposed upstream of the first relaxing wheel in the feed direction of the composite web.

The cutting station is configured to make a succession of lines of weakness on the composite web between a second closing element and a first closing element of the absorbent sanitary article. The first closing element follows next after the second closing element in the feed direction of the composite web. Each line of weakness is made and disposed between consecutive absorbent articles in the feed direction of the composite web. Each absorbent sanitary article comprises the first closing element at one end of the first flap and the second closing element at one end of the second flap.

In the case where the forming apparatus comprises a cutting station upstream of the rotary device and the rotary device is a folding wheel, the folding wheel, while folding, can also separate the absorbent articles by tearing the composite web at the lines of weakness.

Preferably, the cutting station comprises a cutting wheel round which the composite web is wrapped. The lines of weakness may be made by an electronically cammed cutter acting in conjunction with the cutting wheel.

The computerized unit may be configured to slow the relaxing wheel relative to the cutting wheel so that the composite web slows down on the relaxing wheel, downstream of the cutting wheel, thus modifying the spacing of the absorbent articles until bringing them to the working spacing of the working units on the rotary device.

The rotary device may be a welding wheel for welding the absorbent sanitary articles in an apparatus for forming pant style absorbent articles of the type described above. The working units each comprise at least one welder operating on the composite web.

The rotary device may be configured to weld a continuous strip of absorbent articles, in composite web form, to weld the side gathers of pant style nappies.

An example of a rotary device for welding pant style nappies is described in application WO2020/165749A1, which is incorporated herein by reference for completeness of description.

The composite web is a continuous strip of absorbent articles, in particular a continuous strip of nappy pants known as “training pants”.

The continuous strip is a succession of absorbent articles folded along the centre line of the absorbent pad. The composite web material is preferably held on the wheels and/or on the rotary device by suction and/or belts and the like may be used.

### BRIEF DESCRIPTION OF THE DRAWINGS

Further features and advantages of the aforesaid aspects are more apparent in the exemplary, hence non-limiting description of a preferred but non-exclusive embodiment of a method and an apparatus for forming absorbent sanitary articles.

The description is set out below with reference to the accompanying drawings which are provided solely for purposes of illustration without restricting the scope of the invention and in which:

FIGS. 1 to 9 are plan views schematically illustrating a succession of steps of a forming method according to the description;

FIG. 10 is a schematic front view illustrating a forming apparatus according to this description;

FIG. 11 is a schematic front view illustrating a forming apparatus according to this description;

FIG. 12 is a plan view illustrating a composite web which can be processed with the forming apparatus of FIG. 11.

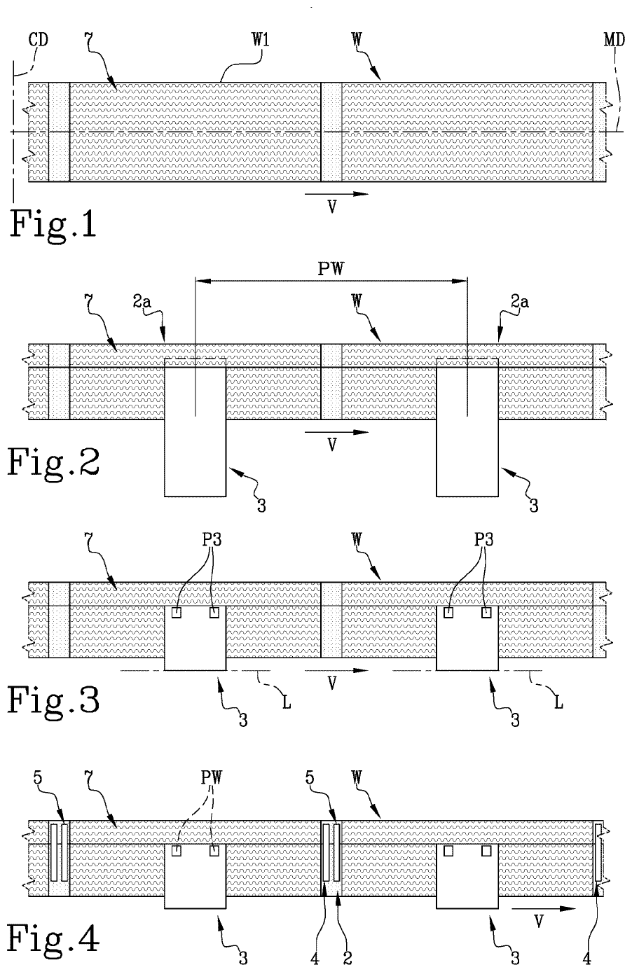

### DETAILED DESCRIPTION OF PREFERRED EMBODIMENTS OF THE INVENTION

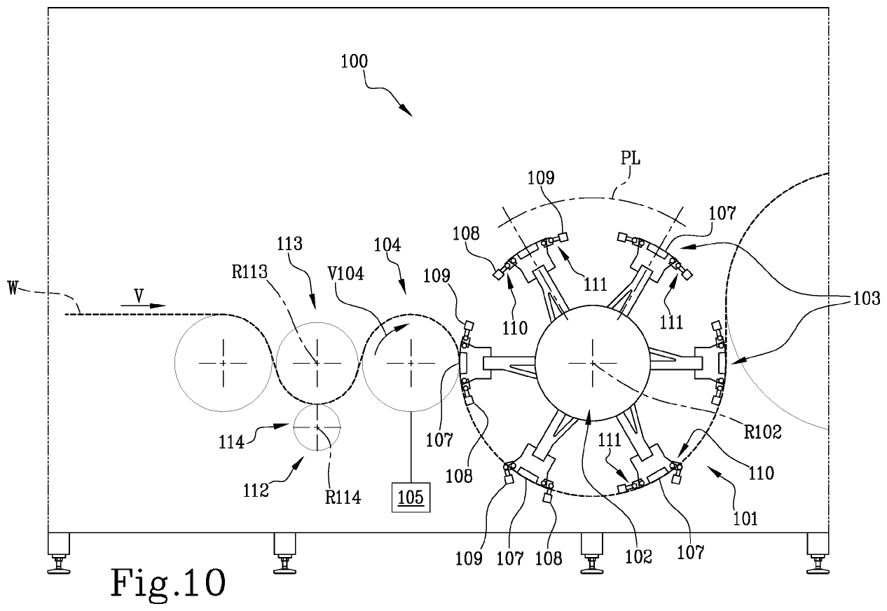

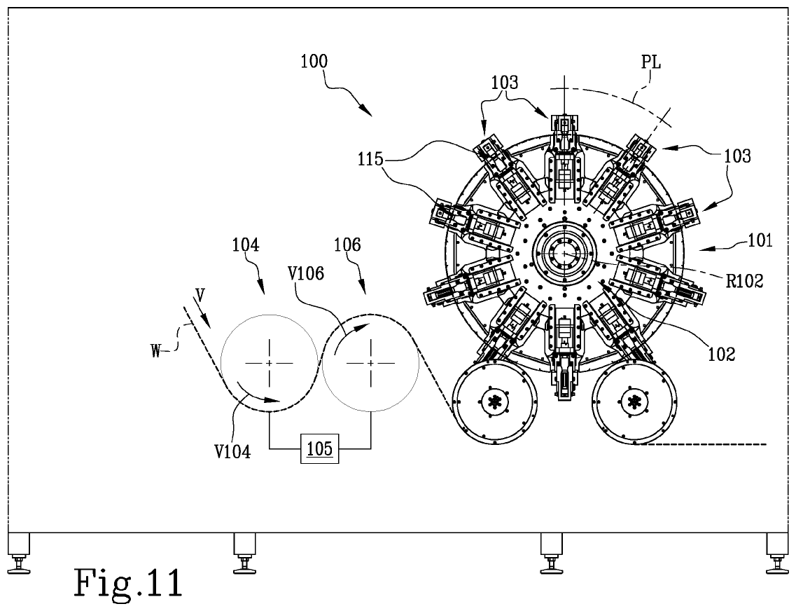

With reference to FIGS. 10 and 11, the numeral 100 denotes an apparatus for forming absorbent sanitary articles.

The apparatus 100 is intended for forming an absorbent sanitary article from an elasticized composite web W.

The web W, of substantially known type, is called “composite” in that it comprises a plurality of components which are joined to each other and which are described only insofar as necessary for understanding this invention.

The web W is elasticized at least in a feed direction V of the web W itself along an advancing direction MD (Machine Direction).

The web W comprises, for example, a first and a second web of non-woven fabric, joined to each other, and a plurality of elastics 7 interposed between them. At least part of these elastics is illustrated and labelled in the accompanying drawings. The elastics 7 are preferably spread across the full width of the web W. The elastics 7 substantially determine the elasticity of the web W.

The first and the second web may, for example, be glued and/or welded to each other, for example by ultrasounds, in substantially known manner.

In an embodiment, illustrated in FIG. 10, the apparatus 100 is intended for forming nappies of the type also known as “refastenable” nappies which, when open, are basically in the shape of a T.

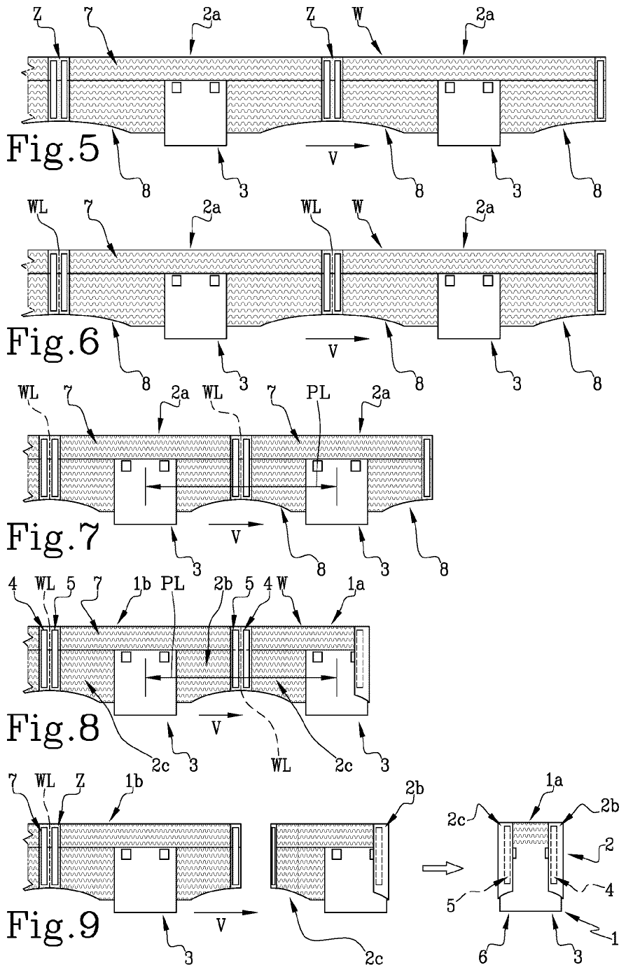

An example of a wearable absorbent sanitary article of the type known as “refastenable” is denoted by the reference numeral 1 in FIG. 9.

The nappy 1 essentially comprises an elastic waistband or waist gather 2 and an absorbent pad 3 attached to the elastic waistband 2.

The absorbent pad 3 is attached to an intermediate portion 2a of the elastic waistband 2 from which it projects, when the nappy 1 is open, to form the T-shaped structure.

The elastic waist gather is the horizontal portion of the T and the absorbent pad 3 constitutes the stem of the T.

In the elastic waistband 2, the absorbent pad 3 separates two portions, or wings, or side flaps 2b, 2c.

The article 1 comprises a closing element 4 attached, at one end of the flap 2b, to the face of the flap 2b which, in use, is directed towards a wearer (not illustrated).

The article 1 comprises a closing element 5 attached, at one end of the flap 2c, to the face of the flap 2c which, in use, is directed towards a wearer (not illustrated).

The closing elements 4 and 5 are of substantially known type and, in the example illustrated, extend along the corresponding edge of the respective flap.

When the nappy 1 is being used or during its manufacture, the absorbent pad 3 is folded towards the waistband 2 to define a crotch 6 of the article 1.

At least in these cases, the flaps 2b, 2c are folded onto the folded absorbent pad 3 and attached thereto by the closing element 4 and the closing element 5 respectively.

In embodiments not illustrated, the nappy 1 may comprise a closing element attached to the absorbent pad on the side opposite that of the wearer so that the closing elements 4, 5 of the elastic waistband engage the closing element on the pad.

As illustrated schematically in FIG. 9, the nappy 1 in the closed configuration is substantially pant-like.

The composite web W comprises a succession of absorbent pads 3 of corresponding absorbent sanitary articles 1. The pads 3 are disposed along a direction CD (Cross Direction) substantially orthogonal to the feed direction of the composite web W and to the direction MD.

With reference to FIGS. 2 to 6, the pads 3 and the corresponding absorbent articles 1 have a spacing PW along the composite web W, that is, each pad 3 is spaced from the preceding one and the next one by the spacing PW.

The apparatus 100 comprises a rotary device 101 essentially comprising a rotary means 102 and a plurality of working units 103 configured to perform a process on the composite web W.

For example, a process may comprise folding the first flap of each absorbent article towards the corresponding absorbent pad and folding the second flap of each absorbent article towards the corresponding absorbent pad, may comprise separating the individual absorbent sanitary articles from the composite web, may comprise folding the individual absorbent sanitary articles, may comprise welding the composite web, may comprise welding the elasticized gathers which encircle the waist of the wearer of the pant style nappy.

The working units 103 are configured to perform a process on the composite web W at least partly wrapped round the rotary device 101 while the rotary means 102 rotates about an axis of rotation R102.

The working units 103 are supported by the rotary means 102 and are angularly spaced from each other about the axis R102.

The angular distance between a first working unit 103 and a second working unit 103 adjacent to it determines the working spacing PL of the rotary device 101 on the composite web W.

The forming apparatus 100 comprises a relaxing wheel 104 for relaxing the web W and located upstream of the rotary device 101 in the feed direction V of the web W.

The wheel 104 is provided with suction means and the web W is at least partly wrapped round it.

The forming apparatus 100 comprises a computerized command and control unit, represented schematically as a block 105, in communication with the relaxing wheel 104.

The computerized unit 105 is configured to adjust a rotation speed V104 of the wheel 104 as a function of the working spacing PL of the rotary device 101 so that the composite web W is shortened on the wheel 104 until the pads 3 (and the corresponding absorbent sanitary articles 1) are spaced at the working spacing PL of the rotary device 101.

In practice, the relaxing wheel 104 allows bringing the pads 3 from the spacing PW to the spacing PL.

The relaxing wheel 104 decreases the speed of the web W relative to the speed of the web W itself upstream of the relaxing wheel 104.

By slowing the web on the wheel 104, the elastics 7 of the composite web W relax and/or are shortened so that the absorbent articles 1 withdraw naturally, thus modifying the spacing of the products being processed.

In the example of FIG. 11, the forming apparatus 100 comprises a second relaxing wheel 106 between the relaxing wheel 104 and the rotary device 101. The wheel 106 is located downstream of the wheel 104 upstream of the rotary device 101 in the feed direction V of the web W.

The wheel 106 is provided with suction means and the web W is at least partly wrapped round it.

The computerized unit 105 is in communication with the relaxing wheel 106 and is configured to adjust the rotation speed V106 of the wheel 106 as a function of the working spacing PL of the rotary device 101.

The computerized unit 105 is configured to adjust the rotation speeds V104, V106 of the relaxing wheels 104, 106 so that the composite web W is shortened on the wheel 104 and on the wheel 106 until the absorbent pads 3 (and the corresponding absorbent sanitary articles 1) are spaced at the working spacing PL of the rotary device 101.

The speed V106 of the wheel 106 is lower than the speed V104 of the wheel 104 so that the speed of the web W, hence the spacing between the absorbent articles 1, is reduced in successive steps.

In alternative embodiments not illustrated, the forming apparatus 100 comprises more than two relaxing wheels based on the overall slowing to be imparted to the composite web W and to the expected precision of the spacing of the absorbent articles 1 on the rotary device 101.

In the example of FIG. 10, the rotary device 101 is a folding wheel for folding the absorbent sanitary articles 1.

Basically, each working unit 103 comprises a main support 107 for receiving a respective absorbent pad 3 of the succession of absorbent pads 3 in the web W.

Each working unit 103 comprises a first and a second side support 108, 109 disposed on opposite sides of the main support 107 to receive, respectively, the first and the second flap 2b, 2c of the waistband 2 of the absorbent article 1 corresponding to the absorbent pad 3 received by the main support 107.

The first and the second flap 2b, 2c extend on opposite sides of the respective absorbent pad 3. Each working unit 103 comprises a first and a second folder 110, 111 for folding the first side support 108 and the second side support 109 on the main support 107 respectively.

In the embodiment illustrated, the apparatus 100 comprises a cutting station 112 for cutting the web W and located upstream of the relaxing wheel 104 in the feed direction V of the web W.

The cutting station 112 is configured to make a succession of lines of weakness WL or score lines or the like in the web W between each closing element 5 and the closing element 4 following next after the closing element 5 in the feed direction V of the composite web W. Each line of weakness WL is made and disposed between consecutive absorbent articles 1 in the feed direction V of the composite web W.

The station 112 comprises, for example, a suction wheel 113 which is rotatable about an axis of rotation R113 and round which the web W is wrapped.

The station 112 comprises a rotary cutter 114, preferably electronically cammed, so as to follow any spacing between the lines of weakness WL along the web W. The cutter 114 is rotatable about an axis of rotation R114.

The folding wheel shown in FIG. 10 can separate the absorbent articles 1 from each other, while they are being folded, by tearing the web W at the lines of weakness WL.

The computerized unit 105 is configured to drive the relaxing wheel 104 relative to the cutting wheel 113 so that the web W slows down on the relaxing wheel 104.

The wheel 104 is set in rotation at a lower speed compared to the wheel 113.

Slowing the web W downstream of the wheel 113 modifies the spacing between the absorbent articles 1 to bring it to the preset spacing corresponding to the spacing PL of the working units 103 on the rotary device 101.

In an embodiment, illustrated in FIG. 11, the apparatus 100 is intended for forming nappies of the type also known as pants, training pants or pant style.

Pant style nappies are essentially in the shape of an H, where the legs of the H are elasticized and constitute the parts of the nappy that encircle the wearer's waist, while the connecting portion of the H is the crotch portion, provided with an absorbent pad. The elastic waist gathers of the nappy are welded to each other, that is, joined together permanently.

The rotary device 101 is a welding wheel for welding the elastic waist gathers of the absorbent article 1.

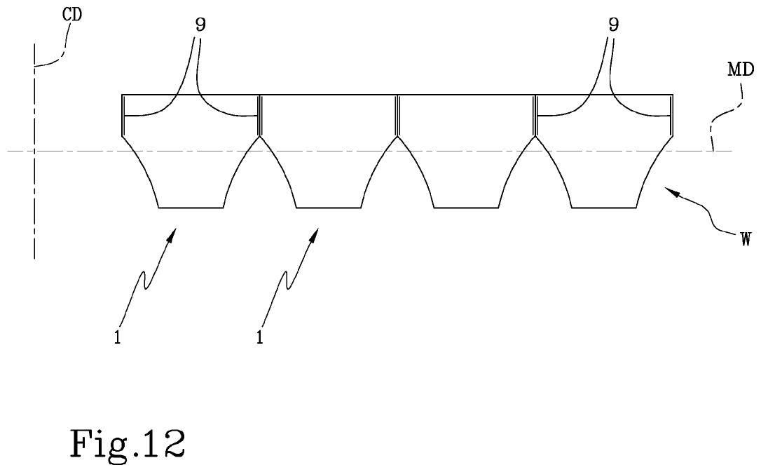

As illustrated schematically in FIG. 12, the composite web W is a continuous strip of absorbent articles 1 folded along the centre line of the absorbent pad 3.

The device 101 is configured to weld the web W along the direction CD and to make a plurality of welds 9, each defining a transverse weld of superposed elastic gathers of respective absorbent articles 1.

The working units 103 comprise welding units 115 of substantially known type.

The welds 9 at the wearer's hips permanently seal the elasticized gathers of the absorbent article 1 which encircle the wearer's waist.

The wheel 104 and/or the wheel 106 upstream of the rotary device 101 allow slowing the web W so as to bring the spacing of the absorbent articles 1, hence of the welds 9, to the working spacing PL of the welding wheel.

The forming method comprises wrapping the composite web W at least partly round the rotary device 101.

The forming method comprises performing a process on the composite web W which is at least partly wrapped round the rotary device 101.

The forming method comprises relaxing the composite web W before wrapping the composite web W round the rotary device 101 so that the composite web W is shortened until the absorbent pads 3 and the corresponding absorbent sanitary articles 1 are spaced at the working spacing PL of the rotary device 101.

The forming method comprises relaxing the composite web W before performing a process on the composite web W so that the composite web W is shortened until the absorbent pads 3 and the corresponding absorbent sanitary articles 1 are spaced at the working spacing PL of the rotary device 101.

The step of relaxing allows adjusting the tension of the web W so that the absorbent articles 1 are brought to the preset spacing PL, which becomes the common spacing for all the sizes to be made in the apparatus 100.

Relaxing the web W comprises slowing the composite web W, for example on the relaxing wheels 104, 106 before wrapping the composite web W round the rotary device 101.

Relaxing the web W comprises slowing the composite web W, for example on the relaxing wheels 104, 106 before performing a process on it.

With reference to FIG. 11, relaxing the composite web comprises a first relaxing (on the wheel 104) and a second relaxing (on the wheel 106) of the composite web W.

With reference to FIG. 10, performing a process comprises separating the individual absorbent sanitary articles 1 from the composite web W.

Performing a process comprises folding the individual absorbent sanitary articles 1.

With reference to FIG. 2, the forming and folding method comprises a step of applying the succession of absorbent pads 3-for example with glue-so they jut outwards and are spaced apart by a spacing which may be a function of the size of the absorbent article 1.

Preferably, the method comprises a step of folding an edge W1 of the web W the upper edge, looking at the drawings-onto the absorbent pads 3.

Next, the absorbent pads 3 are folded onto the web W about a folding line L which, in the example illustrated, is parallel to the web W and to the advancing direction MD of the web W.

The method comprises a step of folding the absorbent pads 3 onto the composite web W and a step of temporarily fastening the folded absorbent pads to the web W.

The absorbent pads 3 are fastened to the web W temporarily in such a way as to remain in a folded configuration, as illustrated in FIG. 3, until the remaining steps of the method are carried out on it.

Preferably, the step of temporarily fastening the folded absorbent pads to the composite web W comprises a step of plastically deforming a portion P3 of the folded absorbent pads 3 and a portion of the composite web W located substantially at the portion P3 to create mechanical interference between the absorbent pads 3 and the composite web W.

The method comprises a step of applying a succession of the first closing elements 4 and a succession of the second closing elements 5 on the composite web W.

As illustrated by way of an example in FIG. 4, each absorbent pad 3 is interposed between a first closing element 4 and a second closing element 5.

In the case where the composite web W is formed of a first web glued to a second web with the elastics 7 interposed between them, the method may comprise a step of cutting or interrupting the elastics 7 in the zones where the closing elements 4, 5 are applied.

For example, the accompanying drawings illustrate zones Z at the closing elements 4, 5, where the elastics 7 have been cut or omitted.

In the case where the composite web W is formed of a first web glued tom a second web with the elastics 7 interposed between them, the method may comprise a step of cutting or interrupting the elastics 7 in the zones where the absorbent pads, 3 are applied.

In the case where the composite web W is formed of a first web welded to a second web with the elastics 7 interposed between them, the method may comprise a step of welding the first web to the second web except at the zones where the closing elements 4, 5 are applied.

In the case where the composite web W is formed of a first web welded to a second web with the elastics 7 interposed between them, the method may comprise a step of welding the first web to the second web except at the zones where the absorbent pads 3 are applied.

With reference to FIG. 5, in the embodiment illustrated by way of example, the method comprises a step of contouring the web W between the absorbent pads 3.

With reference to FIG. 5, it is noted, for example, that the web W comprises contours 8, where portions of the web W have been removed.

The method comprises a step of making a succession of lines of weakness WL in the composite web W between each second closing element 5 and the first closing element 4 following it in the feed direction V, each line of weakness WL comprising, for example, a series of cuts which perforate the web W.

In practice, each line of weakness WL defines an end of the second flap 2c of a first absorbent article 1a and the end of the first flap 2b of a second absorbent article 1b following the first absorbent article 1a in the feed direction V.

Each line of weakness WL separates an absorbent article 1 from the one following it along the web W.

According to the method, the web W is then relaxed so as to modify the spacing of the absorbent articles 1 to bring it to the working spacing PL so that folding and tearing on the rotary device 101 are carried out precisely at the lines of weakness WL.

The forming and folding method comprises a step of folding the first flap 2b of each absorbent article 1 towards the corresponding absorbent pad 3 and a step of folding the second flap 2c of each absorbent article 1 towards the corresponding absorbent pad 3.

Folding the first flap 2b of each absorbent article 1 towards the corresponding absorbent pad 3 or folding the second flap 2c of each absorbent article 1 towards the corresponding absorbent pad 3 tears the composite web W at the lines of weakness WL, thereby separating the absorbent articles 1 from each other.

In the example illustrated, with reference in particular to FIG. 9, it is by folding the second flap 2c that the absorbent web W is torn at the lines of weakness WL.

In the embodiment of the method schematically represented in the accompanying drawings, the step of folding the first flap 2b of the absorbent article 1 towards the corresponding absorbent pad 3 is carried out before the step of folding the second flap 2c of the absorbent article 1 towards the corresponding absorbent pad 3.

In an embodiment of the method not illustrated, the step of folding the first flap 2b of the absorbent article 1 towards the corresponding absorbent pad 3 is carried out after the step of folding the second flap 2c of the absorbent article 1 towards the corresponding absorbent pad 3.

In an embodiment of the method not illustrated, the step of folding the first flap 2b of the absorbent article 1 towards the corresponding absorbent pad 3 is carried out at the same time as the step of folding the second flap 2c of the absorbent article 1 towards the corresponding absorbent pad 3.

According to this method, the step of folding the first flap 2b of the absorbent article 1 towards the corresponding absorbent pad 3 comprises a step of attaching the first flap 2b to the corresponding absorbent pad 3 with the first closing element 4.

According to this method, the step of folding the second flap 2c of the absorbent article 1 towards the corresponding absorbent pad 3 comprises a step of attaching the second flap 2c to the corresponding absorbent pad 3 with the second closing element 5.

With reference to FIG. 11, performing a process comprises welding the composite web W.

Performing a process comprises welding the elastic gathers which encircle the waist of the wearer of the pant style nappy.

The forming method may comprise preparing the composite web W and feeding it in the feed direction.

1. A method for forming an absorbent sanitary article made from a composite web elasticized at least along a machine direction of the composite web and comprising a succession of absorbent pads of corresponding absorbent sanitary articles, the forming method comprising:
relaxing the composite web before wrapping the composite web round the rotary device
so that the composite web is shortened until the absorbent pads of the absorbent sanitary articles and the corresponding absorbent sanitary articles are spaced at a working spacing of the rotary device;
performing a process on the composite web at least partly wrapped round a rotary device.

2. The forming method according to claim 1, wherein relaxing the composite web comprises slowing down the composite web.

3. The forming method according to claim 1, wherein performing a process comprises separating the individual absorbent sanitary articles from the composite web.

4. The forming method according to claim 1, wherein performing a process comprises folding the absorbent sanitary articles.

5. The forming method according to claim 1, wherein the absorbent sanitary article comprises:
a waistband,
the absorbent pad extending from an intermediate portion of the waistband and separating a first and a second flap of the waistband,
a first closing element at one end of the first flap, and
a second closing element at one end of the second flap, the absorbent pad being, at least in use, folded towards the waistband to define a crotch of the absorbent article, and the first and second flaps (2b, 2c) being folded onto the folded absorbent pad and fixed thereto by the first closing element and the second closing element, respectively, to define pants, the forming method comprising:
applying the succession of absorbent pads on the composite web so they are spaced from each other and project outwards therefrom;
folding the absorbent pads onto the composite web;
temporarily fastening the folded absorbent pads to the composite web;
applying a succession of the first closing elements on the composite web;
applying a succession of the second closing elements on the composite web, each absorbent pad being interposed between one of the first closing elements and one of the second closing elements
the forming method comprising:
making a succession of lines of weakness on the composite web between each second closing element and the next first closing element following the second closing element in a feed direction of the composite web before relaxing the composite web or during relaxing of the composite web,
each line of weakness being made and positioned between consecutive absorbent sanitary articles in the feed direction,
performing a process on the composite web comprising:
folding the first flap of each absorbent article towards the corresponding absorbent pad;
folding the second flap of each absorbent article towards the corresponding absorbent pad,
folding the first flap of each absorbent article towards the corresponding absorbent pad or folding the second flap of each absorbent article towards the corresponding absorbent pad so as to tear the composite web at the lines of weakness and to separate the absorbent articles.

6. The forming method according to claim 5, wherein folding the first flap of the absorbent article towards the corresponding absorbent pad is carried out before folding the second flap of the absorbent article towards the corresponding absorbent pad or is carried out after folding the second flap of the absorbent article towards the corresponding absorbent pad or is carried out at the same time as folding the second flap of the absorbent article towards the corresponding absorbent pad

7. The forming method according to claim 4, wherein folding the first flap of the absorbent article towards the corresponding absorbent pad comprises attaching the first flap to the corresponding absorbent pad with the first closing element and/or attaching the second flap to the corresponding absorbent pad with the second closing element

8. The forming method according to claim 4, wherein temporarily fastening the folded absorbent pads to the composite web comprises plastically deforming at least one portion of each folded absorbent pad and one portion of the composite web at the portion of each folded absorbent pad to create mechanical interference between the absorbent pads and the composite web.

9. The forming method according to claim 1, wherein relaxing the composite web is carried out in at least two distinct steps and comprises a first relaxing of the composite web and a second relaxing of the composite web after the first relaxing of the composite web.

10. The forming method according to claim 1, wherein performing a process comprises welding the composite web.

11. An apparatus for forming an absorbent sanitary article made from a composite web elasticized at least along a machine direction of the composite web in the forming apparatus and comprising a succession of absorbent pads of corresponding absorbent sanitary articles,
the forming apparatus comprising:
a rotary device comprising a rotary means rotating in a feed direction of the composite web and a plurality of working units configured to perform a process on the composite web at least partly wrapped round the rotary device as the rotary means rotates about an axis of rotation, the working units being supported by the rotary means and being disposed angularly spaced from each other around the axis of rotation of the rotary means, an angular distance between a first working unit and a second working unit adjacent thereto determining a working spacing of the rotary device on the composite web, the forming apparatus comprising:
at least one first relaxing wheel for relaxing the composite web and disposed upstream of the rotary device,
a computerized command and control unit in communication with the first relaxing wheel,
the computerized command and control unit being configured to adjust a rotation speed of the first relaxing wheel as a function of the working spacing of the rotary device so that the composite web is shortened on the first relaxing wheel until the absorbent pads of the absorbent sanitary articles and the corresponding absorbent sanitary articles are spaced at the working spacing of the rotary device.

12. The forming apparatus according to claim 11, comprising a second relaxing wheel between the first relaxing wheel and the rotary device, the computerized command and control unit being in communication with the second relaxing wheel and being configured to adjust a rotation speed of the second relaxing wheel as a function of the working spacing of the rotary device so that the composite web is shortened on the first and the second relaxing wheel, until the absorbent pads of the absorbent sanitary articles and the corresponding absorbent sanitary articles are spaced at the working spacing of the rotary device

13. The apparatus according to claim 11, wherein each working unit comprises a main support for receiving a respective absorbent pad of the succession of absorbent pads, a first and a second side support disposed on opposite sides of the main support to receive, respectively, a first and a second flap of a waistband of the absorbent sanitary article corresponding to the absorbent pad received by the main support, the first and the second flap extending on opposite sides of the respective absorbent pad, each working unit comprising a first and a second folder, for folding the first side support and the second side support on the main support respectively, the absorbent sanitary article comprising the waistband, the absorbent pad extending from an intermediate portion of the waistband separating the first and the second flap of the waistband.

14. The apparatus according to claim 11, comprising a cutting station for cutting the composite web, disposed upstream of the first relaxing wheel in the feed direction of the composite web AAA and configured to make a succession of lines of weakness on the composite web between a second closing element and a first closing element of the absorbent sanitary article, the first closing element following next after the second closing element in the feed direction of the composite web, each line of weakness being made and disposed between consecutive absorbent sanitary articles in the feed direction, the absorbent sanitary article comprising the first closing element at one end of the first flap and the second closing element at one end of the second flap, the absorbent pad being, at least in use, folded towards the waistband to define a crotch of the absorbent article, and the first and second flaps being folded onto the folded absorbent pad and fixed thereto by the first closing element and the second closing element respectively, to define pants.

15. The apparatus according to claim 11, wherein the rotary device is a welding wheel for welding the absorbent sanitary articles and the working units each comprise at least one welding unit operating on the composite web.
